(19) United States
(12) Patent Application Publication
(10) Pub. No.: US 2010/0292487 A1
(43) Pub. Date: Nov. 18, 2010

(54) CHEMICAL PROCESS FOR THE PREPARATION OF BENZOXAZOLE DERIVATIVES USED AS PESTICIDES

(75) Inventor: Martin Diggelmann, Berkshire (GB)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA LIMITED, Bracknell, Berkshire (GB)

(21) Appl. No.: 12/063,615

(22) PCT Filed: Aug. 15, 2006
(86) PCT No.: PCT/GB2006/003054

§ 371 Date: Jul. 27, 2010

(30) Foreign Application Priority Data
Aug. 19, 2005 (GB) 0517051.9

(51) Int. Cl. C07D 417/12

(52) U.S. Cl. 548/214

(57) ABSTRACT

This invention relates to a process for the preparation of compounds of formula (I): where R^{a}, R^{b}, R¹, R², R³, R⁴, R⁵, R⁶, R⁷, R⁸, R⁹, and R¹⁰ are defined organic groups, the process comprising reaction a compound of formula (II): with a compound of formula (III): where R^{c} is as defined in relation to formula (I) followed by treatment with a base and cyclising the resulting adduct.

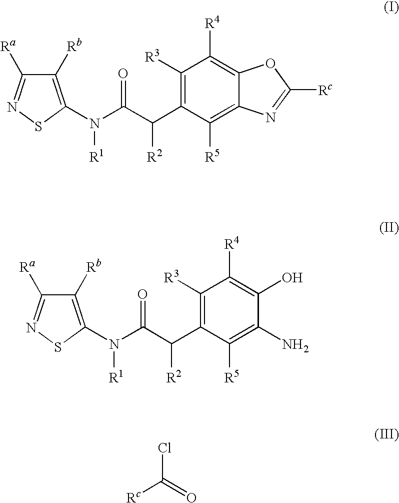

The present invention relates to an improved process for making azole derivatives useful as insecticidal, acaricidal, molluscicidal and nematicidal compounds.

Azole derivatives with useful insecticidal properties are disclosed in WO00/06566, WO00/63207, WO01/55144 and WO03/011861. The applicants have found a method of making the compounds in improved yield and purity. There is therefore provided a process for the preparation of compounds of formula (I)

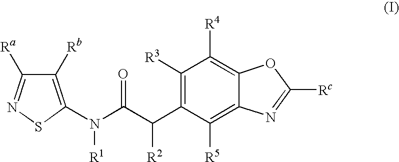

wherein R^{a} is C₁₋₃alkyl; R^{b} is halogen; R^{c} is C₁₋₆ alkoxy(C₁₋₆)alkyl, C₁₋₆ haloalkyl, C₁₋₆ alkyl, C₁₋₆ alkoxy, furfuryl or is a group

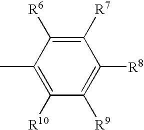

R¹ is hydrogen, C₁₋₂ alkyl, (C₁₋₆)alkoxymethyl or propargyl; R² is hydrogen, methyl or fluoro; R³, R⁴ and R⁵ are, independently, hydrogen, halogen, C₁₋₂ alkyl, C₁₋₂ alkoxy or C₁₋₂ haloalkyl; R⁶ and R¹⁰ are, independently, hydrogen, halogen, C₁₋₃ alkyl, C₁₋₂ haloalkyl, C₁₋₂ alkoxy, nitro, cyano, C₁₋₂ haloalkoxy, C₁₋₈ alkylthio, C₁₋₆ alkylsulfinyl, C₁₋₆ alkylsulfonyl, amino, C₁₋₃ alkylamino or di(C₁₋₃)alkylamino; R⁷, R⁸ and R⁹ are, independently, hydrogen, halogen, C₁₋₆ alkyl, C₂₋₆ alkenyl, C₂₋₆ alkynyl, C₁₋₆ haloalkyl, C₁₋₆ alkoxy(C₁₋₆)alkyl, C₁₋₆ alkoxy, C₁₋₆ alkoxy(C₁₋₆)alkoxy, C₂₋₆ alkynyloxy, C₃₋₆ cycloalkyl, nitro, cyano, C₁₋₆ haloalkoxy, C₂₋₆ haloalkenyloxy, S(O)ₚR¹¹, OSO₂R¹², NR¹³SO₂R¹⁴, NR¹⁵R¹⁶, NR¹⁷COR¹⁸, COR¹⁹, SiR²⁰R²¹R²², SCN, optionally substituted aryl or optionally substituted heteroaryl or optionally substituted heterocyclyl; R¹¹, R¹² and R¹⁴ are, independently, C₁₋₆ alkyl, C₁₋₆ haloalkyl or optionally substitituted aryl; R¹³ and R¹⁷ are, independently, hydrogen or C₁₋₂ alkyl; R¹⁵ and R¹⁶ are, independently, hydrogen or C₁₋₃ alkyl; or R¹⁵ and R¹⁶ together with the N atom to which they are attached form a five or six-membered optionally substituted heterocyclic ring which may contain a further heteroatom selected from O and S; R¹⁸ and R¹⁹ are, independently, hydrogen, C₁₋₆ alkyl, C₁₋₆ alkoxy, optionally substituted aryl, optionally substituted heteroaryl or NR²³R²⁴; R²⁰, R²¹ and R²² are, independently, C₁₋₄ alkyl or aryl; R²³ and R²⁴ are, independently, hydrogen or C₁₋₃ alkyl; or R²³ and R²⁴ together with the N atom to which they are attached form a five or six-membered optionally substituted heterocyclic ring which may contain a further heteroatom selected from O and S; and p is 0, 1 or 2, the process comprising reacting a formula of compound II

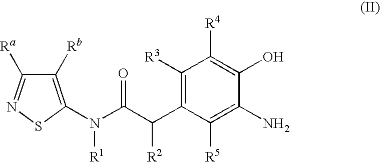

where R^{a}, R^{b}, R¹, R², R³, R⁴ and R⁵ are as defined in relation to formula (I) with a compound of formula III

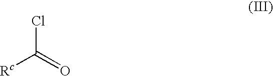

where R^{c} is as defined in relation to formula (I) followed by treatment with a base and cyclising the resulting adduct.

The reaction proceeds via an adduct of formula IV

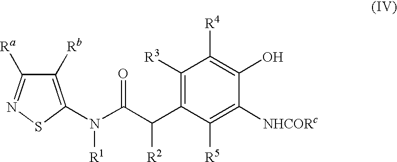

The intermediate of formula (IV) may be isolated or the process can be performed without isolation of the intermediate.

Certain compounds of formula (IV) are novel and as such form a further aspect of the invention.

Suitable conditions for the reactions are described in WO03/011861

The coupling reaction is preferably carried out at −20° C. to 30° C.

The reaction is preferably performed in a solvent. A very wide range of solvents may be used, for example suitable solvents include dimethylacetamide, THF, DMF or DCM.

The preferred molar ratio of acid chloride to aminophenol is from 1:1 to 1:2.

The coupling reaction is preferably carried out in the presence of a base, especially a tertiary amine.

The further treatment with a base may be with any suitable base such as an amine, preferably a primary amine or inorganic bases. A preferred base is ammonia.

Suitable conditions for the cyclisation reaction are described in WO03/011861. Suitable solvents are chloralkanes such as 1,1,2,2-tetrachlorethane or aromatic hydrocarbons such as toluene or xylene.

The acylation reaction reaction between II and III is very difficult to control in order to avoid diacylation i.e. there is an undesirable acylation of the hydroxy group of II as well as the desired acylation of the amino group of II. The applicants have surprisingly found that the further treatment with bases produces compounds of sufficiently high purity such that no further purification is required.

Each alkyl moiety is a straight or branched chain and is, for example, methyl, ethyl, n-propyl, n-butyl, n-pentyl, n-hexyl, iso-propyl, n-butyl, sec-butyl, iso-butyl, tert-butyl or neo-pentyl.

Halogen is fluorine, chlorine, bromine or iodine.

Haloalkyl groups are alkyl groups which are substituted with one or more of the same or different halogen atoms and are, for example, CF₃, CF₂Cl, CF₃CH₂ or CHF₂CH₂.

Alkenyl and alkynyl moieties can be in the form of straight or branched chains. The alkenyl moieties, where appropriate, can be of either the (E)- or (Z)-configuration. Examples are vinyl, allyl, ethynyl and propargyl.

Haloalkenyl moieties are alkyl moieties which are substituted with one or more of the same or different halogen atoms, an example being CH₂CH═CCl₂.

Aryl includes naphthyl, anthracyl, fluorenyl and indenyl but is preferably phenyl.

The term heteroaryl refers to an aromatic ring containing up to 10 atoms including one or more heteroatoms (preferably one or two heteroatoms) selected from O, S and N. Examples of such rings include pyridine, pyrimidine, furan, quinoline, quinazoline, pyrazole, thiophene, thiazole, oxazole and isoxazole.

The terms heterocycle and heterocyclyl refer to a non-aromatic ring containing up to 10 atoms including one or more (preferably one or two) heteroatoms selected from O, S and N. Examples of such rings include 1,3-dioxolane, tetrahydrofuran and morpholine.

Cycloalkyl includes cyclopropyl, cyclopentyl and cyclohexyl.

When present, the optional substituents on aryl, heteroaryl or heterocyclyl are selected, independently, from hydrogen, halogen, C₁₋₆ alkyl, C₂₋₆ alkenyl, C₂₋₆ alkynyl, C₁₋₆ haloalkyl, C₁₋₆ alkoxy(C₁₋₆)alkyl, C₁₋₆ alkoxy, C₃₋₆ cycloalkyl, nitro, cyano, C₁₋₆ haloalkoxy, C₁₋₂ alkylthio, SO₂CH₃, SO₂CH₂CH₃, OSO₂CH₃ and SCN.

It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected (C₁₋₆)alkyl groups. When heterocyclic rings are formedby joining two groups on an N atom, the resulting rings are suitably pyrrolidine, piperidine, thiomorpholine and morpholine each of which may be substituted by one or two independently selected (C₁₋₆) alkyl groups.

Preferred groups for R^{a}, R^{b}, R^{c}, R¹, R², R³, R⁴ and R⁵ in any combination thereof are set out below.

Preferably R^{a} is methyl or ethyl.

It is preferred that R^{b} is bromo or chloro, especially chloro.

The group R^{c} is preferably is a group

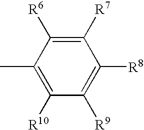

or is C₁₋₆ alkyl or is C₁₋₆ haloalkyl.

More preferably R^{c} is C₁₋₆ alkyl or C₁₋₆ haloalkyl, more especially C₁₋₃ haloalkyl.

Preferably R¹ is hydrogen, C₁₋₂ alkyl or (C₁₋₆) alkoxymethyl.

It is more preferred that R¹ is hydrogen, ethyl, CH₂OCH₃ or CH₂OC₂H₅.

Yet more preferably R¹ is hydrogen, ethyl or CH₂OC₂H₅.

It is even more preferred that R¹ is hydrogen or CH₂OC₂H₅, especially hydrogen.

Preferably R² is hydrogen or fluoro.

In one aspect of the invention, it is preferred that R² is fluoro.

Preferably R³, R⁴ and R⁵ are each, independently, hydrogen or halogen.

It is preferred that R³ is hydrogen or fluorine.

More preferably R³ is hydrogen.

It is preferred that R⁴ is hydrogen or fluorine.

More preferably R⁴ is hydrogen.

It is preferred that R⁵ is hydrogen or fluorine.

More preferably R⁵ is hydrogen.

It is preferred that R⁷, R⁸ and R⁹ are each, independently, hydrogen, halogen, C₁₋₆ alkyl, C₁₋₆ haloalkyl, C₁₋₆ alkoxy, C₁₋₆ alkoxy(C₁₋₆)alkoxy, C₂₋₆ alkynyloxy, nitro, cyano, C₁₋₆ alkylthio, C₁₋₆ alkylsulfonyl or C₂₋₆ haloalkenyloxy.

It is preferred that R⁷ is hydrogen, halogen, C₁₋₆ alkyl, C₁₋₆ alkoxy(C₁₋₆)alkoxy, nitro or cyano.

More preferably R⁷ is hydrogen, chlorine, fluorine, methyl, OC₂H₄OCH₃, nitro or cyano.

It is even more preferred that R⁷ is hydrogen or chlorine.

It is yet more preferred that R⁷ is hydrogen.

It is preferred that R⁸ is hydrogen, halogen, C₁₋₆ haloalkyl, C₁₋₆ alkoxy, C₁₋₆ alkoxy(C₁₋₆)alkoxy, C₂₋₆ alkynyloxy, cyano, C₁₋₆ alkylsulfonyl or C₂₋₆ haloalkenyloxy.

More preferably R⁸ is hydrogen, chlorine, fluorine, bromine, CF₃, ethoxy, OC₂H₄OCH₃, OCH₂C₂H, cyano, SO₂CH₃ or OCH₂CH═CCl₂.

It is even more preferred that R⁸ is hydrogen, chlorine, CN, CF₃ or SO₂CH₃.

Yet more preferably R⁸ is hydrogen.

It is preferred that R⁹ is hydrogen, halogen or C₁₋₆ alkylthio.

More preferably R⁹ is hydrogen, chlorine, fluorine, iodine or SCH₃.

It is even more preferred that R⁹ is hydrogen, chlorine or fluorine.

Yet more preferably R⁹ is hydrogen.

It is preferred that R⁶ and R¹⁰ are, independently, hydrogen, halogen, C₁₋₃ alkyl, C₁₋₂ haloalkyl, C₁₋₂ alkoxy, nitro, cyano, C₁₋₂ haloalkoxy, C₁₋₈ alkylthio or C₁₋₆ alkylsulfinyl, C₁₋₆ alkylsulfonyl; provided that at least one of R⁶ and R¹⁰ is not hydrogen.

In one aspect of the invention, it is preferred that R⁶ and R¹⁰ are, independently, hydrogen, halogen, C₁₋₃ alkyl, C₁₋₂ haloalkyl, C₁₋₂ alkoxy, nitro, cyano, C₁₋₂ haloalkoxy or C₁₋₂ alkylthio, provided that at least one of R⁶ and R¹⁰ is not hydrogen.

It is more preferred that R⁶ is hydrogen, methyl, chlorine, fluorine or bromine and R¹⁰ is hydrogen, methyl, chlorine, fluorine, OCH₃, SCH₃, CF₃ or nitro, provided that at least one of R⁶ and R¹⁰ is not hydrogen.

It is still more preferred that R⁶ is hydrogen, chlorine, fluorine or bromine and R¹⁰ is hydrogen, chlorine, fluorine, OCH₃, SCH₃, CF₃ or nitro, provided that at least one of R⁶ and R¹⁰ is not hydrogen.

Even more preferably R⁶ is hydrogen, chlorine, fluorine or bromine and R¹⁰ is chlorine, fluorine or bromine.

It is most preferred that when R⁶ is hydrogen, R¹⁰ is fluorine, chlorine or bromine and that when R⁶ is chlorine or fluorine, R¹⁰ is fluorine.

The invention is illustrated by the following Example:

### EXAMPLE 1

### Step 1

311 mg (1 mmol) of 2-(3-Amino-4-hydroxy-phenyl)-N-(4-chloro-3-ethyl-isothiazol-5-yl)-acetamide was dissolved in 4.5 ml of THF and 417 ul of triethylamine (3 mmol) added. After cooling the solution to 0° degrees, a freshly prepared solution of 168 mg 3-furfuryl acid chloride (1.5 mmol) was added in dropwise fashion under stirring. After addition the icebath was removed and the resulting suspension stirred ambient temperature for another 2 hrs before 1 ml of cone aq. ammonia was added. After 12 hrs the reaction mixture was concentrated to dryness (N2-stream) and consequently worked-up by liquid-liquid extraction with EtOAc/1N HCl. The resulting crude material was used without further purification in the next step.

### Step 2

The crude material was dissolved in 6 ml trichloroethylene, 40 mg (0.2 mmol) p-TsOH added and the resulting suspension heated under stirring to 150 deg overnight. After removal of the solvent the remaining crude material was dissolved in 2 ml DMF and the required product separated via RP-HPLC.

1. A process for the preparation of compounds of formula (I) 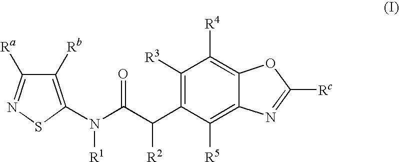
wherein R^{a} is C₁₋₃ alkyl; R^{b} is halogen; R^{c} is C₁₋₆ alkoxy(C₁₋₆)alkyl, C₁₋₆ haloalkyl, C₁₋₆ alkyl, C₁₋₆ alkoxy, furfuryl or is a group 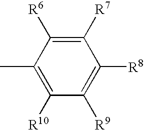
R¹ is hydrogen, C₁₋₂ alkyl, (C₁₋₆)alkoxymethyl or propargyl; R² is hydrogen, methyl or fluoro;
R³, R⁴ and R⁵ are, independently, hydrogen, halogen, C₁₋₂ alkyl, C₁₋₂ alkoxy or C₁₋₂ haloalkyl;
R⁶ and R¹⁰ are, independently, hydrogen, halogen, C₁₋₃ alkyl, C₁₋₂ haloalkyl, C₁₋₂ alkoxy, nitro, cyano, C₁₋₂ haloalkoxy, C₁₋₈ alkylthio, C₁₋₆ alkylsulfinyl, C₁₋₆ alkylsulfonyl, amino, C₁₋₃ alkylamino or di(C₁₋₃)alkylamino; R⁷, R⁸ and R⁹ are, independently, hydrogen, halogen, C₁₋₆ alkyl, C₂₋₆ alkenyl, C₂₋₆ alkynyl, C₁₋₆ haloalkyl, C₁₋₆ alkoxy(C₁₋₆)alkyl, C₁₋₆ alkoxy, C₁₋₆ alkoxy(C₁₋₆)alkoxy, C₂₋₆ alkynyloxy, C₃₋₆ cycloalkyl, nitro, cyano, C₁₋₆ haloalkoxy, C₂₋₆ haloalkenyloxy, S(O)ₚR¹¹, OSO₂R¹², NR¹³SO₂R¹⁴, NR¹⁵R¹⁶, NR¹⁷COR¹⁸, COR¹⁹, SiR²⁰R²¹R²², SCN, optionally substituted aryl or optionally substituted heteroaryl or optionally substituted heterocyclyl;
R¹¹, R¹² and R¹⁴ are, independently, C₁₋₆ alkyl, C₁₋₆ haloalkyl or optionally substituted aryl;
R¹³ and R¹⁷ are, independently, hydrogen or C₁₋₂ alkyl; R¹⁵ and R¹⁶ are, independently, hydrogen or C₁₋₃ alkyl; or R¹⁵ and R¹⁶ together with the N atom to which they are attached form a five or six-membered optionally substituted heterocyclic ring which may contain a further heteroatom selected from O and S; R¹⁸ and R¹⁹ are, independently, hydrogen, C₁₋₆ alkyl, C₁₋₆ alkoxy, optionally substituted aryl, optionally substituted heteroaryl or NR²³R²⁴;
R²⁰, R²¹ and R²² are, independently, C₁₋₄ alkyl or aryl;
R²³ and R²⁴ are, independently, hydrogen or C₁₋₃ alkyl; or R²³ and R²⁴ together with the N atom to which they are attached form a five or six-membered optionally substituted heterocyclic ring which may contain a further heteroatom selected from O and S; and p is 0, 1 or 2 the process comprising reacting a formula of compound II 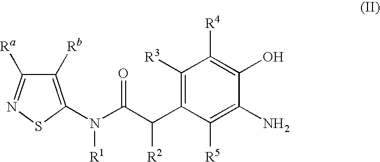
where R^{a}, R^{b}, R¹, R², R³, R⁴ and R⁵ are as defined in relation to formula (I) with a compound of formula III 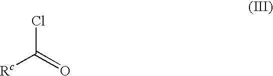
where R^{c} is as defined in relation to formula (I) followed by treatment with a base and cyclising the resulting adduct.

2. A process as claimed in claim 1 where R⁶ and R¹⁹ are, independently, hydrogen, halogen, C₁₋₃ alkyl, C₁₋₂ haloalkyl, C₁₋₂ alkoxy, nitro, cyano, C₁₋₂ haloalkoxy, C₁₋₂alkylthio, amino, C₁₋₃ alkylamino or di(C₁₋₃)alkylamino, provided that at least one of R⁶ and R¹⁹ is not hydrogen; and R⁷, R⁸ and R⁹ are, independently, hydrogen, halogen, C₁₋₆ alkyl, C₂₋₆ alkenyl, C₂₋₆ alkynyl, C₁₋₆ haloalkyl, C₁₋₆ alkoxy(C₁₋₆)alkyl, C₁₋₆ alkoxy, C₃₋₆ cycloalkyl, nitro, cyano, C₁₋₆ haloalkoxy, S(O)ₚR¹¹, OSO₂R¹², NR¹³SO₂R¹⁴, NR¹⁵R¹⁶, NR⁷COR¹⁸, COR¹⁹, SiR²⁰R²¹R²², SCN, optionally substituted aryl or optionally substituted heteroaryl.

3. A process as claimed in claim 1 wherein R^{c} is C₁₋₆ alkyl or C₁₋₆ haloalkyl.

4. A process as claimed in claim 1 where R¹ is hydrogen, C₁₋₂ alkyl or (C₁₋₆) alkoxymethyl.

5. A process as claimed in claim 1 where R² is hydrogen or fluoro.

6. A process as claimed in claim 1 where R³, R⁴ and R⁵ are each, independently, hydrogen or halogen.
